(19) United States
(12) Patent Application Publication
(10) Pub. No.: US 2026/0033874 A1
(43) Pub. Date: Feb. 5, 2026

(54) MULTI-DIAMETER K-WIRE FOR ORTHOPEDIC APPLICATIONS

(71) Applicant: Treace Medical Concepts, Inc., Ponte Vedra, FL (US)

(72) Inventors: John T. Treace, Ponte Vedra Beach, FL (US); Sean F. Scanlan, Jacksonville, FL (US); Michael Stedham, Jacksonville, FL (US)

(21) Appl. No.: 19/354,442

(22) Filed: Oct. 9, 2025

Related U.S. Application Data
(63) Continuation of application No. 18/432,396, filed on Feb. 5, 2024, now Pat. No. 12,440,250.
Application No. 18/432,396 is a continuation of application No. 17/018,422, filed on Sep. 11, 2020, now Pat. No. 11,890,039.
(60) Provisional application No. 62/900,391, filed on Sep. 13, 2019.

(51) Int. Cl. A61B 17/72; A61B 17/84; A61B 17/88

(52) U.S. Cl. CPC A61B 17/7291; A61B 17/848; A61B 17/8875

(57) ABSTRACT

A multi-diameter fixation pin, such as a K-wire can be used during an orthopedic procedure. The multi-diameter fixation pin may include a leading diameter sized for the specific orthopedic procedure being undertaken and/or the size of the bone into which the fixation pin is being inserted. The multi-diameter fixation pin may include a trailing diameter different than the leading diameter. The trailing diameter may be sized for use with a specific sized powered driver. For example, in orthopedic procedures that involve inserting larger diameter pins and smaller diameter pins into bone(s), all pins used in the procedure may have a trailing diameter (e.g., the larger diameter or the smaller diameter) that allows the pins to be inserted with the same powered driver (e.g., the same attachment head) without needing different sized drivers or attachment heads for different sized pins.

### RELATED MATTERS

This application is a continuation of U.S. patent application Ser. No. 18/432,396, filed Feb. 5, 2024, which is a continuation of U.S. patent application Ser. No. 17/018,422, filed Sep. 11, 2020, now U.S. Pat. No. 11,890,039, issued Jan. 17, 2024, which claims the benefit of U.S. Provisional Application No. 62/900,391, filed Sep. 13, 2019. The entire contents of each of these applications are incorporated herein by reference.

### TECHNICAL FIELD

This disclosure relates to orthopedic devices and techniques.

### BACKGROUND

Kirschner wires, or K-wires, are sterilized, sharpened, smooth or threaded metal pins configured for a variety of medical, orthopedic, dental, and plastic surgical procedures, as well as various types of veterinary surgeries. K-wires can be used to hold bone fragments together and/or to provide an anchor for skeletal traction. A K-wire may be driven into bone using a powered or hand drill.

Example applications in which K-wires may be used include procedures for realigning comparatively small bones within in the human body, such as bones in the hand or foot. These bones may be misaligned and require anatomical realignment with the aid of a K-wire during a surgical procedure. For example, one common type of bone deformity is hallux valgus, which is a progressive foot deformity in which the first metatarsophalangeal joint is affected and is often accompanied by significant functional disability and foot pain. The metatarsophalangeal joint is laterally deviated, resulting in an abduction of the first metatarsal while the phalanges adduct. This often leads to development of soft tissue and a bony prominence on the medial side of the foot, which is called a bunion. Surgical intervention may be used to correct a bunion deformity.

### SUMMARY

In general, this disclosure is directed to multi-diameter fixation pins for orthopedic procedures, such as multi-diameters K-wires, and techniques utilizing such a fixation pin. A multi-diameter fixation pin can be inserted into a bone during an orthopedic procedure, such as inserted through one bone portion into an adjacent bone portion to fixate the two bone portions relative to each other. The two bone portions may be different portions of the same bone (e.g., separated by a cut or fracture) or different two different bones. In either case, a multi-diameter fixation pin according to the disclosure may include a leading diameter sized for the specific orthopedic procedure being undertaken and/or the size of the bone into which the fixation pin is being inserted. The multi-diameter fixation pin may also include a trailing diameter different than the leading diameter. The trailing diameter may be sized for use with a specific sized powered driver. For example, in orthopedic procedures that involve inserting larger diameter pins and smaller diameter pins into bone(s), all pins used in the procedure may have a trailing diameter (e.g., the larger diameter or the smaller diameter) that allows the pins to be inserted with the same powered driver (e.g., the same attachment head) without needing different sized drivers or attachment heads for different sized pins.

As an example implementation, a clinician may select a powered driver and/or a collet for the powered driver that is configured to receive a certain size fixation pin or certain range of sizes of fixation pins. For example, the clinician may from select one collet for the powered driver from a set of different collets, each of which is sized to receive a different size fixation pin (or range of sizes). The clinician can insert the selected collet into the powered driver. The clinician can then use the powered driver with selected collet to insert multiple fixation pins into one or more bone portions during a procedure.

The clinician can insert a fixation pin having a size acceptable for use with the selected collet into the powered driver and then use the powered driver to drive the fixation pin into one or more bone portions. The fixation pin can have a constant (e.g., same) diameter over it length, e.g., optionally with narrowing or other shaping features adjacent the tip. Before or after inserting the fixation pin with constant diameter into the powered driver and then into a bone portion, the clinician may also wish to insert a fixation pin into a bone portion having a diameter outside of the size acceptable for use with the selected collet. Traditionally, this would require the clinician to obtain a different powered driver or change the collet on the powered driver, necessitating additional time and procedural steps.

A multi-diameter fixation pin according to some examples of the present disclosure can be configured with a driver engagement portion and a bone insertion portion. The driver engagement portion can have a diameter different (smaller or larger) than the bone insertion portion. The driver engagement portion may have a size corresponding to the size of one or more other constant diameter fixation pins used during the surgical procedure. The driver engagement portion of the multi-diameter fixation pin can have a size acceptable for use with the selected collet of the powered driver that is appropriately sized for receiving and driving one or more other constant diameter fixation pins. However, the bone insertion portion of the multi-diameter fixation pin can have a different diameter. As a result, the clinician can insert the multi-diameter fixation pin into the powered driver without changing drivers and/or collets used for inserting one or more other fixation pins during the procedure and insert a portion of the multi-diameter fixation pin into one or more bone portions that has a size not otherwise acceptable for use with the collet and/or driver.

In one example, a method is described that includes inserting a pin having a first diameter into a collet of a powered driver and driving the pin into at least one of a metatarsal and a cuneiform. The method further involves moving the metatarsal relative to the cuneiform to establish a moved position of the metatarsal. The method also includes inserting a driver engagement portion of a multi-diameter fixation pin into the collet of the driver and driving a bone insertion portion of the multi-diameter fixation pin through the metatarsal and into another bone to hold the moved position of the metatarsal. The example specifies that the collet of the powered driver is sized to receive the pin having the first diameter and the driver engagement portion of the multi-diameter fixation pin but not the bone insertion portion of the multi-diameter fixation pin.

In another example, a method is described that includes inserting a driver engagement portion of a multi-diameter fixation pin into a collet of a powered driver and driving a bone insertion portion of the multi-diameter fixation pin through one or more bone portions. The example specifies that the collet of the powered driver is sized to receive the driver engagement portion of the multi-diameter fixation pin but not the bone insertion portion of the multi-diameter fixation pin.

In another example, a surgical fixation wire is described. The surgical fixation wire has a body extending from a first end to a second end opposite the first end. The body defines a driver engagement portion having a first diameter and a bone insertion portion having a second diameter less than the first diameter. The example specifies that the first end of the body defines a tip configured for insertion into a bone and the driver engagement portion is configured for insertion into a powered driver.

The details of one or more examples are set forth in the accompanying drawings and the description below. Other features, objects, and advantages will be apparent from the description and drawings, and from the claims.

### BRIEF DESCRIPTION OF DRAWINGS

FIGS. 1A and 1B are front views of a foot showing a normal first metatarsal position and an example frontal plane rotational misalignment position, respectively.

FIGS. 2A and 2B are top views of a foot showing a normal first metatarsal position and an example transverse plane misalignment position, respectively.

FIGS. 3A and 3B are side views of a foot showing a normal first metatarsal position and an example sagittal plane misalignment position, respectively.

FIG. 4 is an illustration of an example multi-diameter fixation pin according to disclosure.

FIG. 5 is an illustration of an example powered driver system that can be used to drive the multi-diameter fixation pin of FIG. 4.

FIG. 6 is a side perspective view of an example bone positioning guide that can be used with a multi-diameter fixation pin.

FIG. 7 is a top plan view of an example bone preparation guide that can be used with a multi-diameter fixation pin.

FIG. 8 is a perspective view of an example bone preparing guide, spacer, and tissue removing instrument check member that can be used with a multi-diameter fixation pin.

FIG. 9 is a perspective view of a foot depicting a bone positioning guide on the foot prior to an alignment of a first metatarsal.

FIG. 10 is a perspective view of a foot depicting a bone positioning guide on the foot.

FIG. 11 is a perspective view of a foot depicting a bone positioning guide on the foot after an alignment of a first metatarsal and with a spacer inserted into a joint space.

FIG. 12 is a perspective view of a foot depicting a bone preparation guide positioned on the foot.

FIG. 13 is a perspective view of a foot depicting a bone preparation guide on the foot with pins inserted through the bone preparation guide.

FIG. 14 is a perspective view of a foot depicting a removal of a bone preparation guide.

FIG. 15 is a side perspective view of a foot depicting bone plates across a joint between first and second bones.

### DETAILED DESCRIPTION

In general, this disclosure is directed to fixation pins used during orthopedic procedures and techniques utilizing such fixation pins. As used herein, the terms “fixation pin” and “fixation wire” are generally used interchangeably. In practice, clinicians may generally refer to fixation pins having a comparatively small diameter (e.g., 1.6 mm or less) as a fixation wire and fixation pins having a comparatively larger diameter is just a fixation pin. However, the specific nomenclature choice between pin and wire can vary between clinicians and providers. Accordingly, multi-diameter fixation pins as described herein can also embrace multi-diameter fixation wires. Example embodiments of the fixation pins include multi-diameter Kirschner wires, or K-wires, and multi-diameter Steinmann pins.

A multi-diameter fixation pin as described herein can be used in any desired orthopedic procedure, including procedures utilizing only a single fixation pin or procedures utilizing multiple fixation pins (which may all be multi-diameter fixation pins as described herein or a combination of multi-diameter fixation pins and constant diameter fixation pins). The multi-diameter fixation pin can be inserted into a single bone portion or into multiple bone portions when used during the orthopedic procedure. For example, the multi-diameter fixation pin may be inserted through one bone portion and into an adjacent bone portion. The two bone portions may be different portions of the same bone (e.g., separated by a cut or fracture). Alternatively, the two bone portions may be different bones, such as to adjacent bones separated by a joint space.

In an example implementation, a multi-diameter fixation pin can be used during a surgical joint realignment procedure. For example, a multi-diameter fixation pin can be used during a surgical procedure, such as a bone alignment, osteotomy, fusion procedure, and/or other procedures where one or more bones are to be prepared (e.g., cartilage or bone removal and/or cut). Such a procedure can be performed, for example, on bones (e.g., adjacent bones separated by a joint or different portions of a single bone) in the foot or hand, where bones are relatively smaller compared to bones in other parts of the human anatomy. In one example, a procedure utilizing a multi-diameter fixation pin can be performed to correct an alignment between a metatarsal (e.g., a first metatarsal) and a second metatarsal and/or a cuneiform (e.g., a medial, or first, cuneiform), such as in a bunion correction surgery. An example of such a procedure is a Lapidus procedure (also known as a first tarsal-metatarsal fusion). In another example, the procedure can be performed by modifying an alignment of a metatarsal (e.g., a first metatarsal). An example of such a procedure is a basilar metatarsal osteotomy procedure.

A multi-diameter fixation pin may be used with one or more other instruments, such as a bone positioning guide and/or a bone preparation guide. The bone preparation guide may be pinned by one or more fixation pins to one or more bone portions to facilitate preparation of adjacent bone portions for fusion. The bone positioning guide may be used to impart a force to one bone portion to move the bone portion relative to another bone portion.

In some examples, one or more multi-diameter fixation pins are used during a realignment procedure in which a metatarsal (e.g., first metatarsal) is moved relative to and oppose cuneiform across a tarsal-metatarsal joint to establish a moved position of the metatarsal. One or more constant diameter fixation pins (fixation pins not having a multiple diameter) may be used during the surgical procedure. The constant diameter fixation pin(s) may have a diameter different (e.g., smaller or larger) than the diameter of the bone insertion portion of the multi-diameter fixation pin. Accordingly, use of the multi-diameter fixation pin can allow the clinician to insert a fixation pin having a different diameter than the constant diameter fixation pins into one or more bone portions during the surgical procedure without changing driver hardware.

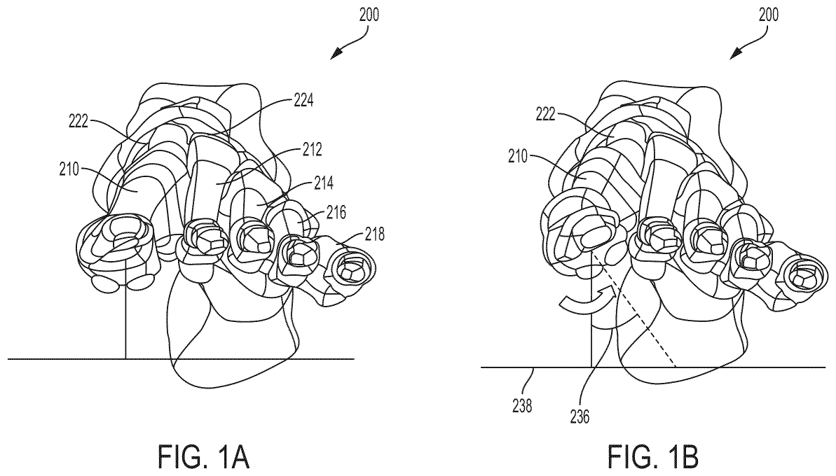

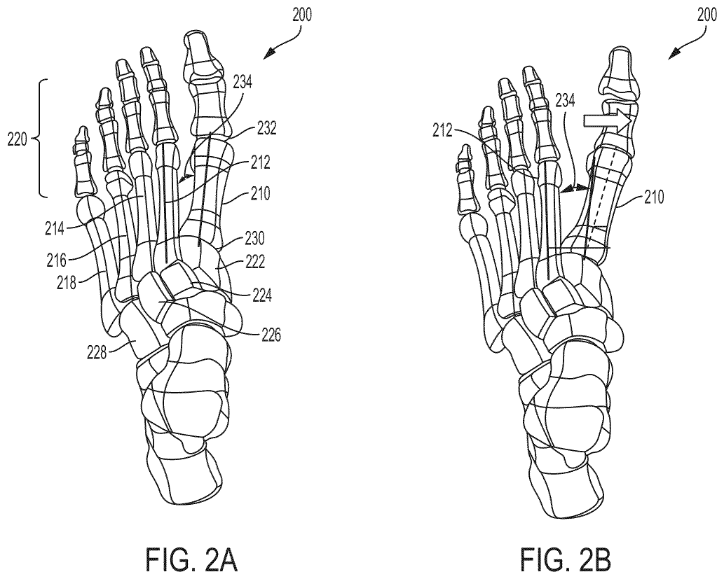

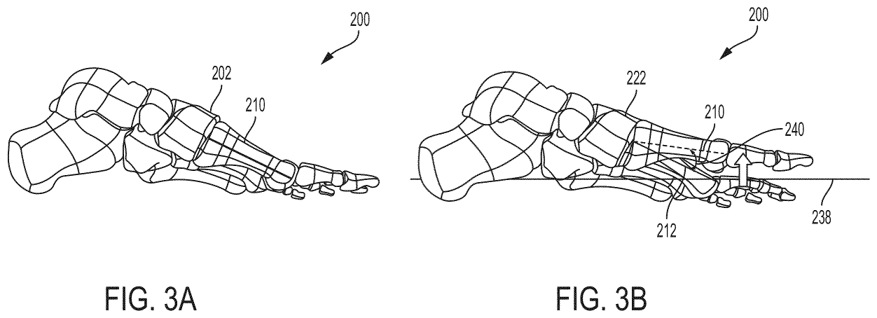

FIGS. 1-3 are different views of a foot 200 showing example anatomical misalignments that may occur and be corrected according to the present disclosure. Such misalignment may be caused by a hallux valgus (bunion), natural growth deformity, or other condition causing anatomical misalignment. FIGS. 1A and 1B are front views of foot 200 showing a normal first metatarsal position and an example frontal plane rotational misalignment position, respectively. FIGS. 2A and 2B are top views of foot 200 showing a normal first metatarsal position and an example transverse plane misalignment position, respectively. FIGS. 3A and 3B are side views of foot 200 showing a normal first metatarsal position and an example sagittal plane misalignment position, respectively. While FIGS. 1B, 2B, and 3B show each respective planar misalignment in isolation, in practice, a metatarsal may be misaligned in any two of the three planes or even all three planes. Accordingly, it should be appreciated that the depiction of a single plane misalignment in each of FIGS. 1B, 2B, and 3B is for purposes of illustration and a metatarsal may be misaligned in multiple planes that is desirably corrected.

With reference to FIGS. 1A and 2A, foot 200 is composed of multiple bones including a first metatarsal 210, a second metatarsal 212, a third metatarsal 214, a fourth metatarsal 216, and a fifth metatarsal 218. The metatarsals are connected distally to phalanges 220 and, more particularly, each to a respective proximal phalanx. The first metatarsal 210 is connected proximally to a medial cuneiform 222, while the second metatarsal 212 is connected proximally to an intermediate cuneiform 224 and the third metatarsal is connected proximally to lateral cuneiform 226. The fourth and fifth metatarsals 216, 218 are connected proximally to the cuboid bone 228. The joint 230 between a metatarsal and respective cuneiform (e.g., first metatarsal 210 and medial cuneiform 222) is referred to as the tarsometatarsal (“TMT”) joint. The joint 232 between a metatarsal and respective proximal phalanx is referred to as a metatarsophalangeal joint. The angle 234 between adjacent metatarsals (e.g., first metatarsal 210 and second metatarsal 212) is referred to as the intermetatarsal angle (“IMA”).

As noted, FIG. 1A is a frontal plane view of foot 200 showing a typical position for first metatarsal 210. The frontal plane, which is also known as the coronal plane, is generally considered any vertical plane that divides the body into anterior and posterior sections. On foot 200, the frontal plane is a plane that extends vertically and is perpendicular to an axis extending proximally to distally along the length of the foot. FIG. 1A shows first metatarsal 210 in a typical rotational position in the frontal plane. FIG. 1B shows first metatarsal 210 with a frontal plane rotational deformity characterized by a rotational angle 236 relative to ground, as indicated by line 238.

FIG. 2A is a top view of foot 200 showing a typical position of first metatarsal 210 in the transverse plane. The transverse plane, which is also known as the horizontal plane, axial plane, or transaxial plane, is considered any plane that divides the body into superior and inferior parts. On foot 200, the transverse plane is a plane that extends horizontally and is perpendicular to an axis extending dorsally to plantarly (top to bottom) across the foot. FIG. 2A shows first metatarsal 210 with a typical IMA 234 in the transverse plane. FIG. 2B shows first metatarsal 210 with a transverse plane rotational deformity characterized by a greater IMA caused by the distal end of first metatarsal 210 being pivoted medially relative to the second metatarsal 212.

FIG. 3A is a side view of foot 200 showing a typical position of first metatarsal 210 in the sagittal plane. The sagittal plane is a plane parallel to the sagittal suture which divides the body into right and left halves. On foot 200, the sagittal plane is a plane that extends vertically and is perpendicular to an axis extending proximally to distally along the length of the foot. FIG. 3A shows first metatarsal 210 with a typical rotational position in the sagittal plane. FIG. 3B shows first metatarsal 210 with a sagittal plane rotational deformity characterized by a rotational angle 240 relative to ground, as indicated by line 238.

A system and technique that utilizes a compressor-distractor and/or a pin lock according to the disclosure can be useful during a bone positioning procedure, for example, to correct an anatomical misalignment of a bones or bones. In some applications, the compressor-distractor can help establish and/or maintain a realignment between a metatarsal and an adjacent cuneiform. Additionally or alternatively, the compressor-distractor can facilitate clean-up and compression between adjacent bone portions between fixation. The pin lock can help hold the compressor-distractor at an appropriate position along the length of a pin or pins connecting the compressor-distractor to bone portions to be compressed and/or distracted.

The metatarsal undergoing realignment may be anatomically misaligned in the frontal plane, transverse plane, and/or sagittal plane, as illustrated and discussed with respect to FIGS. 1-3 above. Accordingly, realignment may involve releasing the misaligned metatarsal for realignment and thereafter realigning the metatarsal in one or more planes, two or more planes, or all three planes. After suitably realigning the metatarsal, the metatarsal can be fixated to hold and maintain the realigned positioned.

While a metatarsal can have a variety of anatomically aligned and misaligned positions, in some examples, the term “anatomically aligned position” means that an angle of a long axis of first metatarsal 210 relative to the long axis of second metatarsal 212 is about 10 degrees or less (e.g., 9 degrees or less) in the transverse plane and/or sagittal plane. In certain embodiments, anatomical misalignment can be corrected in both the transverse plane and the frontal plane. In the transverse plane, a normal IMA 234 between first metatarsal 210 and second metatarsal 212 is less than about 9 degrees. An IMA 234 of between about 9 degrees and about 13 degrees is considered a mild misalignment of the first metatarsal and the second metatarsal. An IMA 234 of greater than about 16 degrees is considered a severe misalignment of the first metatarsal and the second metatarsal. In some embodiments, methods and/or devices according to the disclosure are utilized to anatomically align first metatarsal 210 by reducing the IMA from over 10 degrees to about 10 degrees or less (e.g., to an IMA of 9 degrees or less, or an IMA of about 1-5 degrees), including to negative angles of about-5 degrees or until interference with the second metatarsal, by positioning the first metatarsal at a different angle with respect to the second metatarsal.

With respect to the frontal plane, a normal first metatarsal will be positioned such that its crista prominence is generally perpendicular to the ground and/or its sesamoid bones are generally parallel to the ground and positioned under the metatarsal. This position can be defined as a metatarsal rotation of 0 degrees. In a misaligned first metatarsal, the metatarsal may be axially rotated between about 4 degrees to about 30 degrees or more. In some embodiments, methods and/or devices according to the disclosure are utilized to anatomically align the metatarsal by reducing the metatarsal rotation from about 4 degrees or more to less than 4 degrees (e.g., to about 0 to 2 degrees) by rotating the metatarsal with respect to the adjacent cuneiform.

A multi-diameter fixation pin and technique that utilizes a multi-diameter fixation pin according to the disclosure can be useful during a bone positioning procedure, for example, to correct an anatomical misalignment of a bones or bones. In some applications, the multi-diameter fixation pin can be used to help hold a moved position of a metatarsal relative to an adjacent cuneiform. For example, after a metatarsal is moved relative to an adjacent cuneiform, the multi-diameter fixation pin may be inserted into the move metatarsal and adjacent bone (e.g., through a first metatarsal and into an adjacent second metatarsal). This can provide temporary fixation helping to hold the move position of the metatarsal (e.g., first metatarsal) for further procedural steps, such as applying one or more permanent fixation devices (e.g., plate, screw, intramedullary pin).

The metatarsal undergoing realignment may be anatomically misaligned in the frontal plane, transverse plane, and/or sagittal plane, as illustrated and discussed with respect to FIGS. 1-3 above. Accordingly, realignment may involve releasing the misaligned metatarsal for realignment and thereafter realigning the metatarsal in one or more planes, two or more planes, or all three planes. After suitably realigning the metatarsal, the metatarsal can be fixated to hold and maintain the realigned positioned.

While a metatarsal can have a variety of anatomically aligned and misaligned positions, in some examples, the term “anatomically aligned position” means that an angle of a long axis of first metatarsal 210 relative to the long axis of second metatarsal 212 is about 10 degrees or less (e.g., 9 degrees or less) in the transverse plane and/or sagittal plane. In certain embodiments, anatomical misalignment can be corrected in both the transverse plane and the frontal plane. In the transverse plane, a normal IMA 234 between first metatarsal 210 and second metatarsal 212 is less than about 9 degrees. An IMA 234 of between about 9 degrees and about 13 degrees is considered a mild misalignment of the first metatarsal and the second metatarsal. An IMA 234 of greater than about 16 degrees is considered a severe misalignment of the first metatarsal and the second metatarsal. In some embodiments, methods and/or devices according to the disclosure are utilized to anatomically align first metatarsal 210 by reducing the IMA from over 10 degrees to about 10 degrees or less (e.g., to an IMA of 9 degrees or less, or an IMA of about 1-5 degrees), including to negative angles of about-5 degrees or until interference with the second metatarsal, by positioning the first metatarsal at a different angle with respect to the second metatarsal.

With respect to the frontal plane, a normal first metatarsal will be positioned such that its crista prominence is generally perpendicular to the ground and/or its sesamoid bones are generally parallel to the ground and positioned under the metatarsal. This position can be defined as a metatarsal rotation of 0 degrees. In a misaligned first metatarsal, the metatarsal may be axially rotated between about 4 degrees to about 30 degrees or more. In some embodiments, methods and/or devices according to the disclosure are utilized to anatomically align the metatarsal by reducing the metatarsal rotation from about 4 degrees or more to less than 4 degrees (e.g., to about 0 to 2 degrees) by rotating the metatarsal with respect to the adjacent cuneiform.

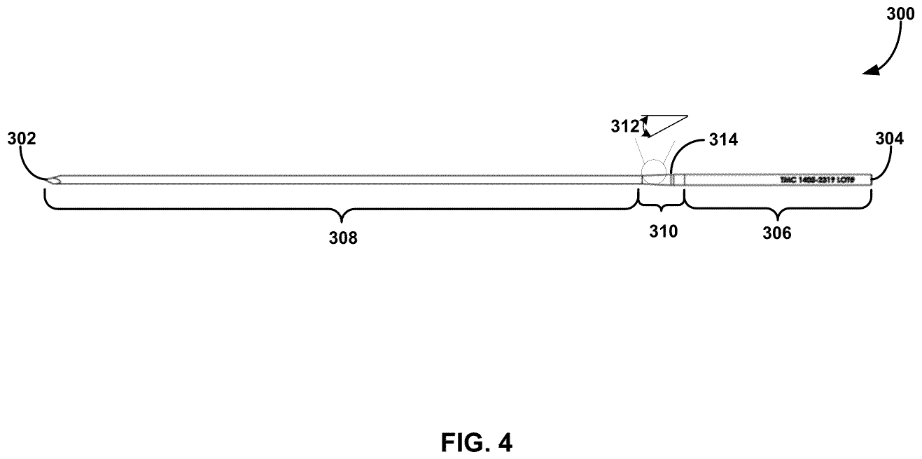

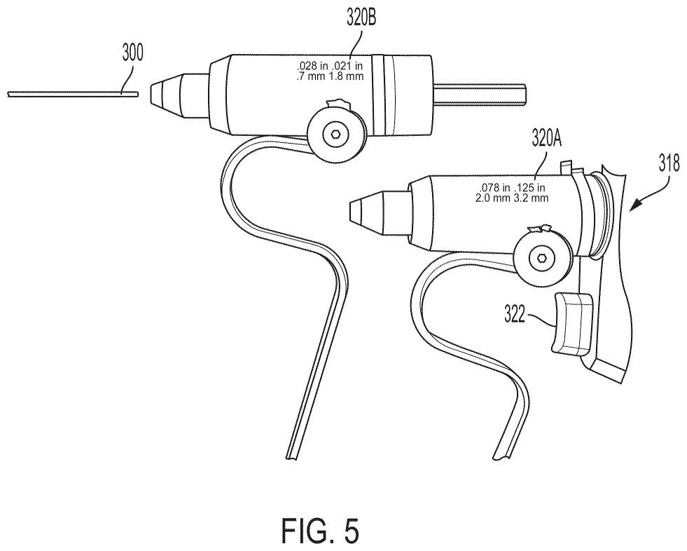

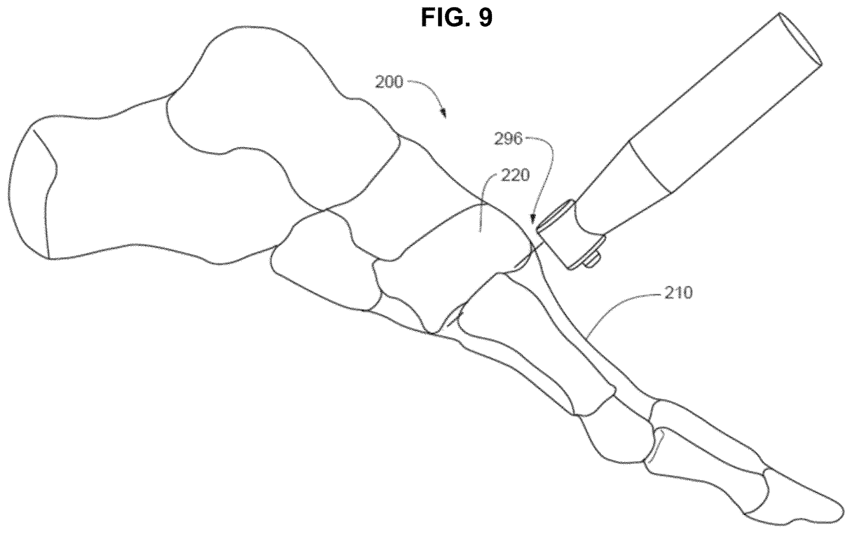

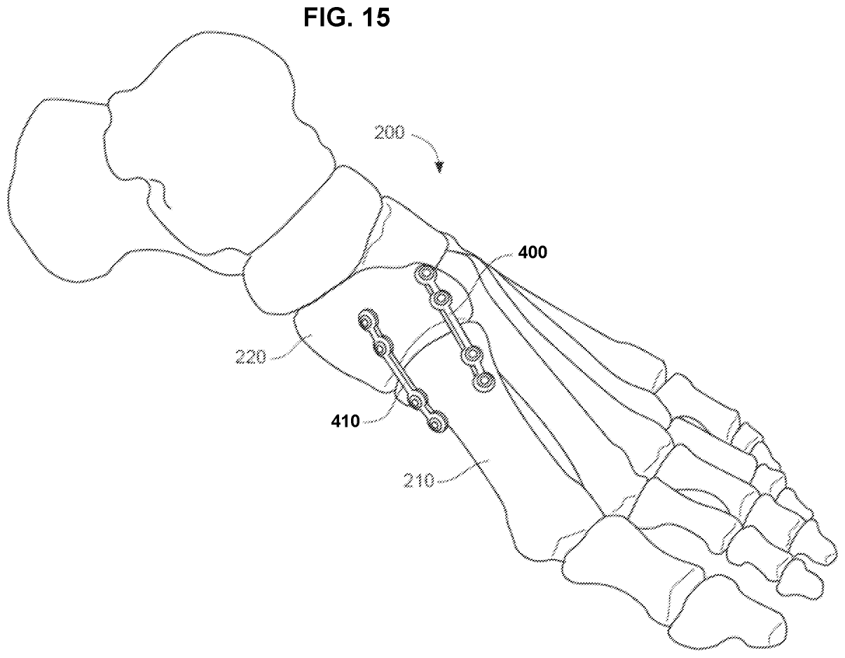

An example technique utilizing a multi-diameter fixation pin will be described in greater detail below with respect to FIGS. 9-15. However, example multi-diameter fixation pin that may be used according to the disclosure will first be described with respect to FIGS. 4 and 5.

FIG. 4 is an illustration of an example multi-diameter fixation pin 300 for an orthopedic procedure. Fixation pin 300 has a body extending lengthwise from a first end 302 to a second and 304 at an opposite end of the body. Fixation pin 300 defines a driver engagement portion 306 and a bone insertion portion 308. Driver engagement portion 306 and bone insertion portion 308 may be different portions of the fixation pin having different cross-sectional areas (e.g., diameters).

For example, driver engagement portion 306 of fixation pin 300 can be configured (e.g., sized and/or shaped) for insertion into a powered driver. The driver engagement portion 306 may have a length and diameter that allows the driver engagement portion to be inserted into a powered driver to a depth appropriate for the powered driver to then act on the fixation pin for driving the pin a surgical procedure

Bone insertion portion 308 a fixation pin 300 can be configured to be partially or fully inserted into one or more bone portions. For example, bone insertion portion 308 may have a diameter different than driver engagement portion 306. In some implementations, bone insertion portion 308 has a diameter smaller than the diameter of driver engagement portion 306. When so configured, a comparatively larger region a fixation pin 300 defined by driver engagement portion 306 can be inserted into the powered driver. A comparatively smaller region a fixation pin 300 defined by bone insertion portion 308 can be partially or fully inserted into and/or through one or more bone portions.

Depending on the procedure being undertaken, it may be clinically beneficial to insert a comparatively smaller diameter fixation pin into the one or more bone portions than a comparatively larger diameter fixation pin. The smaller diameter fixation pin may cause less bone trauma. Additionally or alternatively, in instances where the fixation pin is inserted into comparatively small bones, such as bones of the foot (e.g., cuneiform, first metatarsal, second metatarsal), a comparatively smaller fixation pin may reduce the tendency of the bone(s) to fracture or break upon insertion of a fixation pin. Such fracturing or breaking may occur when a comparatively large fixation pin is inserted into the bone(s).

While fixation pin 300 is described as having a driver engagement portion 306, it should be appreciated that the entire portion need not be inserted into a driver during a surgical procedure. Rather, driver engagement portion 306 may define a section along the length of fixation pin 300 having a first diameter different than one or more other sections of the fixation pin having one or more other diameters. Driver engagement portion 306 may or may not include second end 304.

Likewise, it should be appreciated that while fixation pin 300 is described as having a bone insertion portion 308, the entire portion need not be inserted into a bone during a surgical procedure. Rather, bone insertion portion 308 may define a section along the length of fixation pin 300 having a second diameter different than driver engagement portion 306. At least part of the length of bone insertion portion 308 may be inserted into and/or through one or more bone portions during a surgical procedure.

Fixation pin 300 in some examples, driver engagement portion 306 and bone insertion portion 308 a fixation pin 300 are immediately adjacent to each other along the length of the fixation pin. A sharp (e.g., 90 degree) transition may be provided between driver engagement portion 306 and bone insertion portion 308, such as a dimensional instep or out step between the two differently sized portions. In other configurations, fixation pin 300 to define a taper portion between driver engagement portion 306 and bone insertion portion 308. The taper portion may be a region of dimensional transition between the cross-sectional size of driver engagement portion 306 and the cross-sectional size of bone insertion portion 308.

In the example of FIG. 4, fixation pin 300 is illustrated as including a taper portion 310 sandwiched between driver engagement portion 306 and bone insertion portion 308. Taper portion 310 can have a size (e.g., diameter) that transitions from the cross-sectional size of driver engagement portion 306 to the cross-sectional size of bone insertion portion 308. Taper portion 310 may taper at any suitable angle 312. In some implementations, angle 312 ranges from 5° to 75°, such as from 10° to 60°, or from 15° to 45°.

In some examples, fixation pin 300 may include an indicator 314 positioned at a location along the length of the fixation pin. Indicator 314 may designate how deep fixation pin 300 can be inserted into a powered driver. For example, in use, a clinician may insert second end 304 of fixation pin 300 into a powered driver and advance the fixation pin deeper into the body (e.g., collet) up to indicator. The clinician may not advance fixation pin 300 beyond indicator 314 into the driver. When configured with indicator 314, the indicator may be implemented in a variety of different ways. As one example, a color band or marking may be provided on a perimeter surface of fixation pin 300. As another example, a notch may be formed in a perimeter surface of fixation pin 300. For instance, a laser scribe may be formed partially or fully about a circumferential perimeter of fixation pin 300 to define indicator 314.

As noted, multi-diameter fixation pin 300 includes first end 302. First end 302 is the leading end of the fixation pin that is inserted into bone during a procedure. In some examples, first end 302 is tapered/sharpened to a point or rounded. This may help case insertion of the pin during a surgical procedure. In other examples, first end 302 of fixation pin 300 is not shaped relative to a remainder of bone insertion portion 308.

The specific dimensions of fixation pin 300 can vary depending on the needs of the clinician in the desired procedure in which the fixation pin is going to be utilized. In some examples, driver engagement portion 306 of fixation pin 300 is sized to have a same diameter as one or more other pins used in a surgical procedure (wherein the pins have a constant diameter across their length instead of having multiple diameters). Additionally or alternatively, driver engagement portion 306 of fixation pin 300 may be sized to have a diameter that is different than but sufficiently close to the diameter of one or more other pins used in the surgical procedure such that the fixation pin can be inserted into bone using the same powered driver hardware used to insert the one or more other pins.

Bone insertion portion 308 can have a different diameter (e.g., smaller diameter) than driver engagement portion 306. The diameter of bone insertion portion 308 may be sufficiently different than the diameter of driver engagement portion 306 such that, if the entire length of fixation pin 300 had the diameter of bone insertion portion 308, the fixation pin could not be inserted into bone using the same powered driver hardware used to insert the one or more other pins used in a surgical procedure (wherein the pins have a constant diameter across their length instead of having multiple diameters).

As examples, driver engagement portion 306 may define a first diameter greater than or equal to 2.0 mm, such as a diameter within a range from 2.0 mm to 3.2 mm. Bone insertion portion 308 may define a second diameter less than 2.0 mm, such as a diameter within a range from 0.7 mm to 1.8 mm. Independent of the specific diameters of driver engagement portion 306 and bone insertion portion 308, the difference between the diameters of the two portions may be at least 0.1 mm, such as at least 0.2 mm, or at least 0.5 mm. For example, the difference between the diameters of driver engagement portion 306 and bone insertion portion 308 may range from 0.1 mm to 1.5 mm, such as from 0.2 mm to 0.5 mm.

One or more other pins used in a surgical procedure with fixation pin 300 (wherein the one or more other pins have a constant diameter across their length instead of having multiple diameters) can have a diameter the same as and/or within the same range of as those listed above with respect to driver engagement portion 306. In some examples, the one or more other pins used in the surgical procedure have a diameter that differs from the diameter of driver engagement portion 306 by less than 0.5 mm, such as less than 0.2 mm.

The overall length of multi-diameter fixation pin 300 from first end 302 to second end 304 can also vary depending on the desired clinical application. In different examples, fixation pin 300 may have a length within a range from 25 mm to 500 mm, such as from 50 mm to 260 mm. Further, while fixation pin 300 may typically have a circular cross-sectional shape, the fixation pin can have any polygonal (e.g., square, rectangle) or arcuate (e.g., curved, elliptical) shape.

Fixation pin 300 may be formed as a unitary body from a single piece of material, e.g., via casting, molding, and/or milling. Alternatively, fixation pin 300 may be formed from multiple segments of material (e.g., materials having different diameters) that are then joined together to form a unitary body (e.g., via welding or other permanent joining) technique. In general, fixation pin 300 can be formed from a biocompatible material. Example materials that may be used to fabricate fixation pin 300 include steel (e.g., stainless steel), titanium, ceramic materials, biocompatible plastics, and combinations thereof. After fabrication, fixation pin 300 may be packaged and sterilized, e.g., chemically and/or thermally. Fixation pin 300 may be packaged alone or as part of a kit that includes one or more other pins and/or other instruments used during a surgical procedure.

Multi-diameter fixation pin 300 can be inserted into one or more bone portions using a powered driver. The powered driver may receive the fixation pin by inserting driver engagement portion 306 into the driver and then applying a force to the pin to drive bone insertion portion 308 of the pin into and/or through one or more bone portions. The powered driver may apply a rotary force, causing rotation of the fixation pin, and/or an axial impact force to drive the pin actually into the bone.

FIG. 5 is an illustration of an example system that includes a powered driver 318 that can be used to insert fixation pin 300. Powered driver 318 in this example is shown with interchangeable collets 320A and 320B (collectively referred to as “collet 320”). Collet 320 may be a chuck that forms a collar around an object (e.g., fixation pin 300 and one or more other fixation pins optionally used during a surgical procedure) and can exert a clamping force on the object. Collet 320 can have a sleeve with a cylindrical inner surface. The collet may be squeezed against a matching taper such that its inner surface contracts to a slightly smaller diameter, squeezing a pin inserted therein to hold the pin securely.

Powered driver 318 can have an integral collet 320 that can only receive a specific size or range of sizes of fixation pins. Alternatively, powered driver 318 can have interchangeable collets 320, each of which is configured to receive a different specific size or range of sizes of fixation pins.

Powered driver 318 can be powered by any motive energy, such as a pneumatic energy or electrical energy. To engage powered driver 318, the clinician may depress trigger 322 to drive a fixation pin 300 inserted into the powered driver into a bone. While fixation pin 300 is generally described as being inserted into a bone using a powered driver, such as powered driver 318, it should be appreciated that a fixation pin according to disclosure is not limited to such an example insertion device. For example, the multi-diameter fixation pin can be inserted using hand force or a powered driver having a different configuration than that illustrated and described with respect to FIG. 5.

In some examples, a multi-diameter fixation pin is used as part of a metatarsal realignment procedure in which a metatarsal is realigned relative to an adjacent cuneiform and/or metatarsal in one or more planes, such as two or three planes. Additional details on example bone realignment techniques and devices with which the multi-diameter fixation pin may be used are described in U.S. Pat. No. 9,622,805, titled “BONE POSITIONING AND PREPARING GUIDE SYSTEMS AND METHODS,” filed on Dec. 28, 2015 and issued Apr. 18, 2017, and U.S. Pat. No. 9,936,994, titled “BONE POSITIONING GUIDE,” filed on Jul. 14, 2016 and issued on Apr. 10, 2018, and US Patent Publication No. 2017/0042599 titled “TARSAL-METATARSAL JOINT PROCEDURE UTILIZING FULCRUM,” filed on Aug. 14, 2016. The entire contents of each of these documents are hereby incorporated by reference.

A multi-diameter fixation pin can be used in any desired orthopedic procedure, and the disclosure is not limited to any particular procedure. In some implementations, however, the multi-diameter fixation pin is used as part of a metatarsal realignment procedure. The metatarsal realignment procedure may or may not utilize one or more other surgical instruments, such as a bone positioning guide and/or a bone preparation guide.

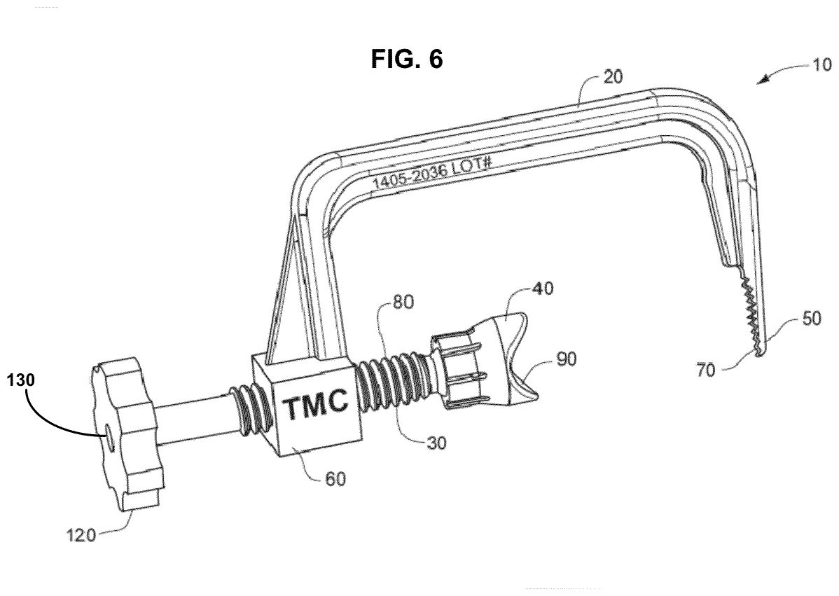

FIG. 6 illustrates an example bone positioning guide 10 that may be used as part of a surgical procedure involving a multi-diameter fixation pin 300. In this example a bone positioning guide 10 includes a main body member 20 and a shaft 30. The bone engagement member 40 is connected to the shaft and a tip 50 is connected to the main body member. In general, the main body member 20 can be sized and shaped to clear anatomy or other instrumentation (e.g., pins and guides) while positioned on a patient. Main body member 20 is illustrated as having a generally C-shaped configuration with a first end 60 and a second end 70.

Shaft 30 can be movably connected to the main body member 20 proximate its first end 60. In some embodiments, the shaft 30 includes threads 80 that engage with the main body member 20 such that rotation of the shaft translates the shaft with respect to the main body member. In other embodiments, the shaft can slide within the main body member and can be secured thereto at a desired location with a set screw. In yet other embodiments, the shaft can be moved with respect to the main body by a ratchet mechanism. In the embodiment shown, the shaft moves along an axis that intersects the tip 50.

Bone positioning guide 10 can include a bone engagement member 40 having a surface 90 configured to contact a bone, such as a metatarsal or a cuneiform. In the embodiment shown, the surface 90 is concave. Such a surface is adapted to promote surface contact with a generally cylindrical bone, such as a metatarsal. In use, bone engagement member 40 can be positioned in contact with a first bone portion (e.g., a first metatarsal) and tip can be positioned in contact with a second bone portion, such as an adjacent bone (e.g., second metatarsal or third metatarsal). Bone positioning guide 10 can include an actuator (e.g., a knob or a handle) 120 to actuate the positioning guide and move bone engagement member 40 relative to tip 50. In the embodiment shown, the actuator can be useful for allowing a user to rotate the shaft with respect to the main body member 20.

In some configurations, bone positioning guide 10 includes a cannulation 130, such as a cannulation 130 extending through the actuator, shaft, and bone engagement member. Multi-diameter fixation pin 300 can be placed through cannulation 130 and into contact with or through a bone engaged with the bone engagement member. For example, fixation wire 300 can be placed into the bone engaged with bone engagement member 40 to fix the position of the bone engagement member with respect to the bone. In another example, fixation wire 300 can be placed through the bone in contact with the bone engagement member and into an adjacent bone (e.g., second metatarsal) to maintain a bone position of the bone in contact with the bone engagement member and the adjacent bone. Fixation pin 300 can be inserted through cannulation 130 and into one or more bone portions using powered driver 318.

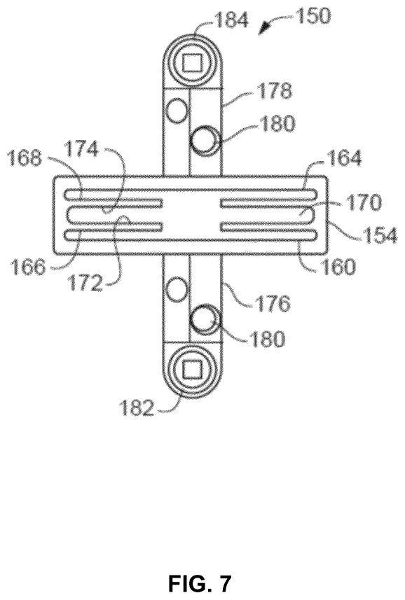

FIG. 7 illustrates an example bone preparation guide 150 that may be used as part of a surgical procedure involving a multi-diameter fixation pin 300. In some examples, bone preparation guide 150 includes a body 154 defining a first guide surface 160 to define a first preparing plane and a second guide surface 164 to define a second preparing plane. A tissue removing instrument (e.g., a saw, rotary bur, osteotome, etc., not shown) can be aligned with the surfaces to remove tissue (e.g., remove cartilage or bone and/or make cuts to bone). The first and second guide surfaces 160, 164 can be spaced from each other by a distance, (e.g., between about 2 millimeters and about 10 millimeters, such as between about 4 and about 7 millimeters). In the embodiment shown, the first and second guide surfaces are parallel, such that cuts to adjacent bones using the guide surfaces will be generally parallel.

In some configurations, as shown in FIG. 7, a first facing surface 166 is positioned adjacent the first guide surface 160 and/or a second facing surface 168 is positioned adjacent the second guide surface 164. In such configurations, the distance between the first guide surface and the first facing surface defines a first guide slot, and the distance between the second guide surface and the second facing surface defines a second guide slot. Each slot can be sized to receive a tissue removing instrument to prepare the bone ends. The first and second slots may be parallel or skewed. In the illustrated example, the facing surfaces each contain a gap, such that the surface is not a single, continuous surface. In other embodiments, the facing surfaces can be a single, continuous surface lacking any such gap.

An opening 170 can be defined by the body 154 between the first and second guide surfaces. The opening can be an area between the guide surfaces useful for allowing a practitioner to have a visual path to bones during bone preparation and/or to receive instruments. In the configuration shown, the opening extends across the body and a distance from a surface 172 opposite of the first facing surface 166 to a surface 174 opposite of the second facing surface 168.

The illustrated bone preparation guide also includes a first end 176 extending from the body 154 in a first direction and a second end 178 extending from the body in a second direction. The second direction can be different than the first direction (e.g., an opposite direction). As shown, each of the first end and the second end can include at least one fixation aperture 180 configured to receive a fixation pin to secure the bone preparation guide to an underlying bone. For example, first end 176 of bone preparation guide 150 may define a first fixation aperture through which a first pin is inserted and the second end 178 of bone preparation guide 150 may define a second fixation aperture through which a second pin is inserted. The first and second pins may be inserted using powered driver 318 and may have a constant diameter across their length. The first end 176 and/or the second end 178 of bone preparation guide 150 may also defined one or more additional fixation apertures that are angled (at a non-zero degree angle) or otherwise skewed relative to the two parallel fixation apertures.

In use, a clinician may insert the two pins (e.g., parallel pins) through fixation apertures 180 and may optionally insert one or more angled pins through the one or more angled fixation apertures. This combination of parallel and angled pins may prevent bone preparation guide 150 from being removed from the underlying bones being worked upon.

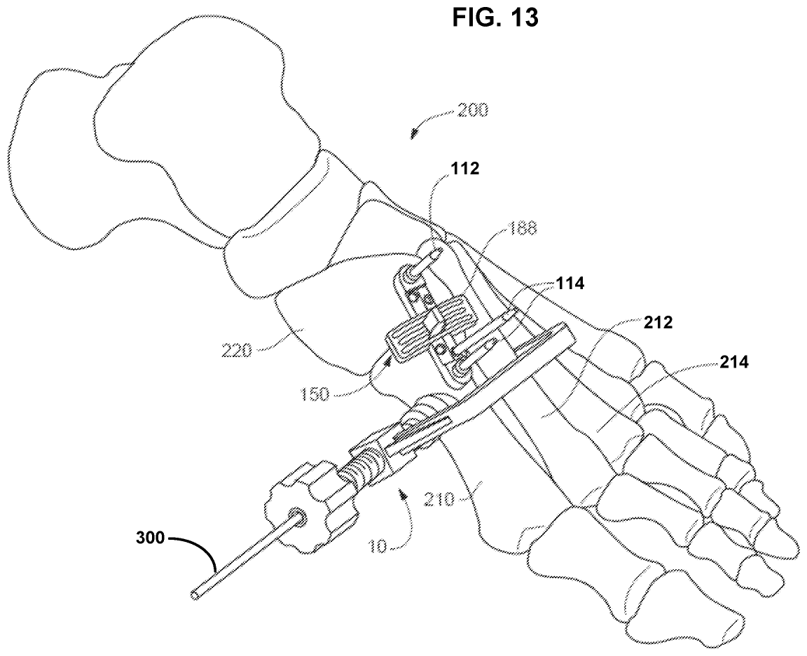

In some examples as shown in FIG. 13, bone preparation guide 150 can also include a first adjustable stabilization member 182 engaged with the first end 176 and/or a second adjustable stabilization member 184 engaged with the second end 178. Each of the members can be threaded and engage a threaded aperture defined by the ends. The elevation of each end can be adjusted with respect to a bone by adjusting the stabilization member. In some embodiments, as shown, the stabilization members are cannulated such that they can receive a fixation pin.

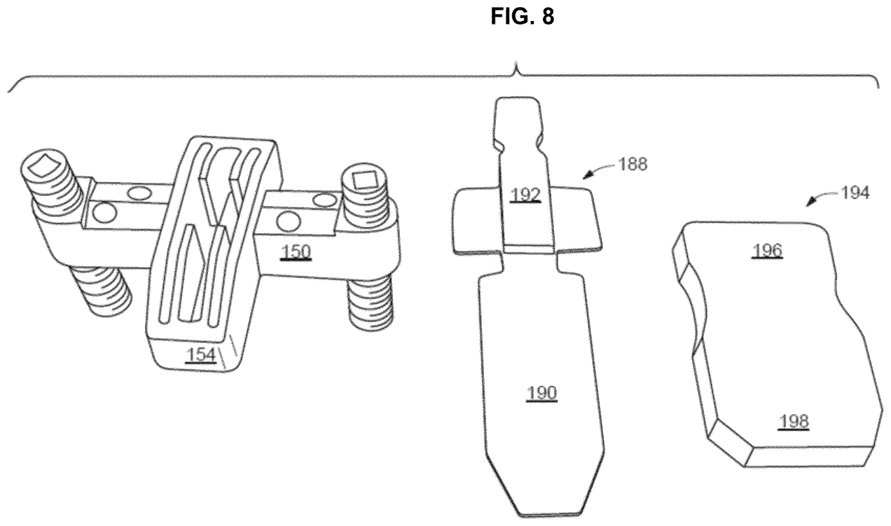

With reference to FIG. 8, bone preparation guide 150 may include or be used with a spacer 188 that extends downward from the body 154. Spacer 188 may be configured to be placed into a joint (e.g., within the TMT joint). In some embodiments, the spacer 188 is selectively engageable with the body of the bone preparation guide and removable therefrom. The spacer can have a first portion 190 configured to extend into a joint space and a second portion 192 engageable with the body 154. In the embodiment shown, the spacer can be received within opening 170, such that the spacer extends from the body in between the first and second guide surfaces. Such a spacer can be useful for positioning the body at a desired position with respect to a joint and for properly positioning the guide with respect to bones to be cut in more than one plane (e.g., three planes selected from more than one of a frontal plane, a transverse plane, and a sagittal plane). The distance between the spacer and the first guide surface can define a length of tissue removal (e.g., bone or cartilage to be cut) from a first bone, and the distance between the spacer and the second guide surface can define a length of tissue removal (e.g., bone or cartilage to be cut) from a second bone.

As also shown in FIG. 8, bone preparation guide 150 may include or be used with a tissue removal location check member 194. Tissue removal check member 194 may be engageable with the body 154 and configured to extend to a first bone and a second bone. The tissue removal location check member can have a first portion 196 configured to extend into contact with first and second bones and a second portion 198 engageable with the body. In the embodiment shown, the tissue removal check member 194 is configured to extend in the body 154 at both the first and second guiding surfaces. The tissue removal location check member 194 may be useful for allowing a practitioner to see where a tissue removing instrument guided by the surfaces will contact the bone to be prepared.

Bone preparation facilitated by bone preparation guide 150 can be useful, for instance, to facilitate contact between leading edges of adjacent bones, separated by a joint, or different portions of a single bone, separated by a fracture, such as in a bone alignment and/or fusion procedure. A bone may be prepared using one or more bone preparation techniques. In some applications, a bone is prepared by cutting the bone. The bone may be cut transversely to establish a new bone end facing an opposing bone portion. Additionally or alternatively, the bone may be prepared by morselizing an end of the bone. The bone end can be morselized using any suitable tool, such as a rotary bur, osteotome, or drill. The bone end may be morselized by masticating, fenestrating, crushing, pulping, and/or breaking the bone end into smaller bits to facilitate deformable contact with an opposing bone portion.

During a surgical technique utilizing fixation pin 300, a bone may be moved from an anatomically misaligned position to an anatomically aligned position with respect to another bone. Further, both the end of the moved bone and the facing end of an adjacent end may be prepared for fixation. In some applications, the end of at least one of the moved bone and/or the other bone is prepared after moving the bone into the aligned position. In other applications, the end of at least one of the moved bone and/or the other bone is prepared before moving the bone into the aligned position.

Movement of one bone relative to another bone can be accomplished using one or more instruments and/or techniques. In some examples, bone movement is accomplished using a bone positioning device that applies a force to one bone at a single location, such that the bone both translates and rotates in response to the force. This may be accomplished, for example, using a bone positioning guide that includes a bone engagement member, a tip, a mechanism to urge the bone engagement member and the tip towards each other, and an actuator to actuate the mechanism. Additionally or alternatively, bone movement may be accomplished using a compressor-distractor. As yet a further addition or alternative, a clinician may facilitate movement by physically grasping a bone, either through direct contact with the bone or indirectly (e.g., by inserting a K-wire such as fixation pin 300, grasping with a tenaculum, or the like), and moving his hand to move the bone.

An example method for preforming a bone alignment procedure utilizing a compressor-distractor and instrument defining a sliding surface according to the disclosure will now be described with respect to FIGS. 9-15 depicting a foot 200 having a first metatarsal 210, a medial cuneiform 222, and a second metatarsal 212. Unless otherwise indicated, the example steps described can be carried out in any order and need not be performed in the order described.

After customary surgical preparation and access, a bone preparation instrument 296 can be inserted into the joint (e.g., first tarsal-metatarsal joint) to release soft tissues and/or excise the plantar flare from the base of the first metatarsal 210, as shown in FIG. 9. Excising the plantar flare may involve cutting plantar flare off the first metatarsal 210 so the face of the first metatarsal is generally planar. This step helps to mobilize the joint to facilitate a deformity correction. In some embodiments, the dorsal-lateral flare of the first metatarsal may also be excised to create space for the deformity correction (e.g., with respect to rotation of the first metatarsal). In certain embodiments, a portion of the metatarsal base facing the medial cuneiform can be removed during this mobilizing step.

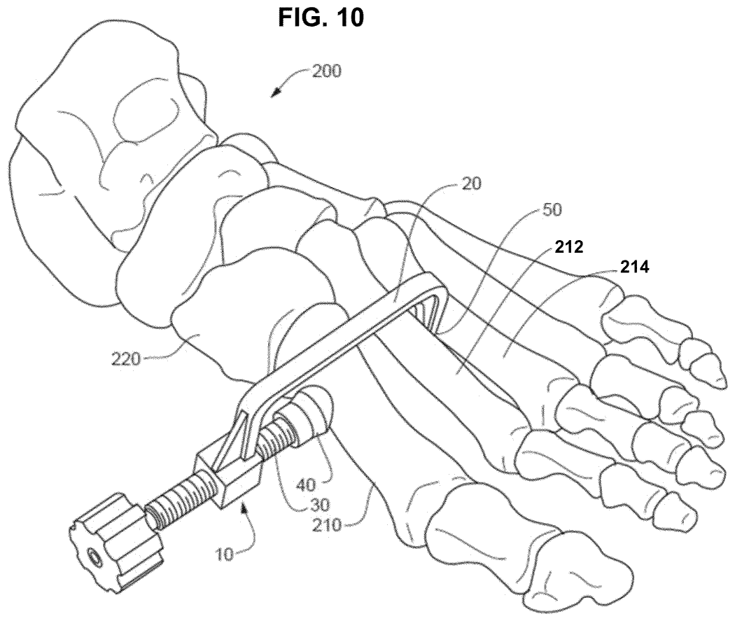

An incision can be made and, if a bone positioning instrument is going to be used, a tip 50 of a bone positioning guide 10 inserted on the lateral side of a metatarsal other than the first metatarsal 210, such as the second metatarsal 212. As shown in FIG. 10, the tip can be positioned proximally at a base of the second metatarsal 212 and a third metatarsal 294 interface. A surface of a bone engagement member 40 can be placed on the proximal portion of the first metatarsal 210. In some embodiments, the bone engagement member engages a medial ridge of the first metatarsal 210. As shown, the body 20 of the positioning guide can be generally perpendicular to the long axis of the second metatarsal 212.

To help avoid a base shift, a clinician can insert a fulcrum in the notch between first metatarsal 210 and second metatarsal 212 at the base of the metatarsals (e.g., adjacent respective cuneiform) before actuating bone positioning guide 10 or otherwise moving the first metatarsal relative to the medial cuneiform. The fulcrum can provide a point about which first metatarsal 210 can rotate and/or pivot while helping minimize or avoid base compression between the first metatarsal and the second metatarsal.

In applications utilizing bone positioning guide 10, the actuator on the bone positioning guide can be actuated to reduce the angle (transverse plane angle between the first metatarsal and the second metatarsal) and rotate the first metatarsal about its axis (frontal plane axial rotation). The first metatarsal 210 can be properly positioned with respect to the medial cuneiform 222 by moving the bone engagement member 40 bone positioning guide with respect to the tip 50 of the bone positioning guide. In some embodiments, such movement simultaneously pivots the first metatarsal with respect to the cuneiform and rotates the first metatarsal about its longitudinal axis into an anatomically correct position to correct a transverse plane deformity and a frontal plane deformity. Other instrumented and/or non-instrumented approaches can be used to adjustment position of first metatarsal 210 relative to medial cuneiform 222. Thus, other applications utilizing compressor-distractor 100 and a pin lock may be performed without utilizing bone positioning guide 10.

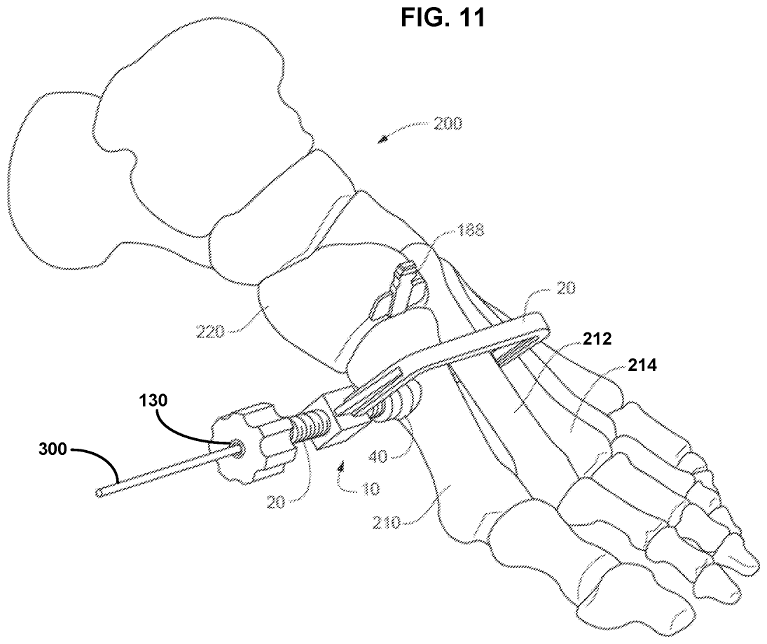

Independent of whether bone positioning guide 10 is used, an example technique may include inserting fixation pin 300 into and through first metatarsal 210 into an adjacent bone, such as second metatarsal 212 and/or medial cuneiform 220. The clinician may insert driver engagement portion 306 of fixation pin 300 into powered driver 318 and use the driver to drive the bone insertion portion of the pin into first metatarsal 210. The clinician may drive bone insertion portion 308 through cannulation 130, through first metatarsal 210, and into second metatarsal 212, as illustrated in FIG. 11. The clinician may detach powered driver 318, leaving fixation pin 300 in the bones to temporarily fixate the moved position of the first metatarsal.

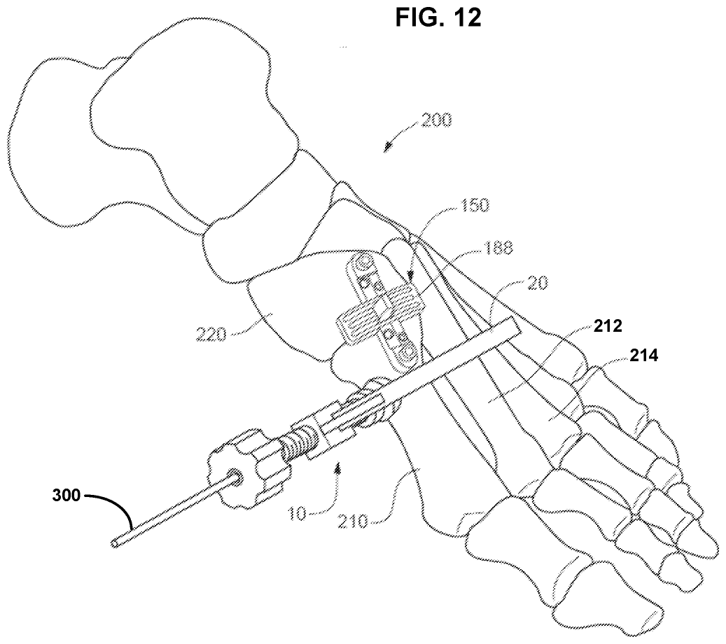

As also illustrated in FIG. 11, the example technique may involve positioning joint spacer 188 within the joint between first metatarsal 210 and medial cuneiform 222. Bone preparation guide 150 can be placed over the joint spacer 188 as shown in FIG. 12 and engaged with the joint spacer to set a position and orientation of the bone preparation guide relative to the joint. In other embodiments, bone preparation guide 150 is placed on the bones without using joint spacer 188 to aid with positioning.

As depicted in FIG. 13, one or more fixation pins can be inserted into apertures of the bone preparation guide 150 to secure the guide to the first metatarsal 210 and the medial cuneiform 222. The fixation pins inserted into the apertures of bone preparation guide 150 includes a first fixation pin 112 and second fixation pin 114. The first and second fixation pins may or may not be multi-diameter fixation pins. For example, the first and second fixation pins may have a constant diameter across their length. The clinician may insert first and second fixation pins 112, 114 using the same driver 318 and same collet 320 used to insert fixation pin 300. One or more additional pins can be inserted at an angle or in a converging orientation to help prevent movement of the bone preparation guide 150 during a tissue removing step. After insertion of the pins, the spacer 188 (if used) can optionally be removed in embodiments having a selectively engageable spacer.

In some applications, the end of the first metatarsal 210 facing the medial cuneiform 222 can be prepared with a tissue removing instrument 296 guided by a guide surface of bone preparation guide 150 (e.g., inserted through a slot defined by a first guide surface and a first facing surface). In some embodiments, the first metatarsal 210 end preparation is done after at least partially aligning the bones, e.g., by actuating bone positioning guide 10 or otherwise moving the first metatarsal but after preparing the end of first metatarsal 210. In other embodiments, the first metatarsal 210 end preparation is done before the alignment of the bones.

In addition to preparing the end of first metatarsal 210, the end of the medial cuneiform 222 facing the first metatarsal 210 can be prepared with the tissue removing instrument 296 guided by a guide surface of bone preparation guide 150 (e.g., inserted through a slot defined by a second guide surface and a second facing surface). In some embodiments, the medial cuneiform 222 end preparation is done after the alignment of the bones. In yet other embodiments, the medial cuneiform 222 end preparation is done before the alignment of the bones. In embodiments that include cutting bone or cartilage, the cuneiform cut and the metatarsal cut can be parallel, conforming cuts. In some examples, a saw blade can be inserted through a first slot to cut a portion of the medial cuneiform and the saw blade can be inserted through a second slot to cut a portion of the first metatarsal.

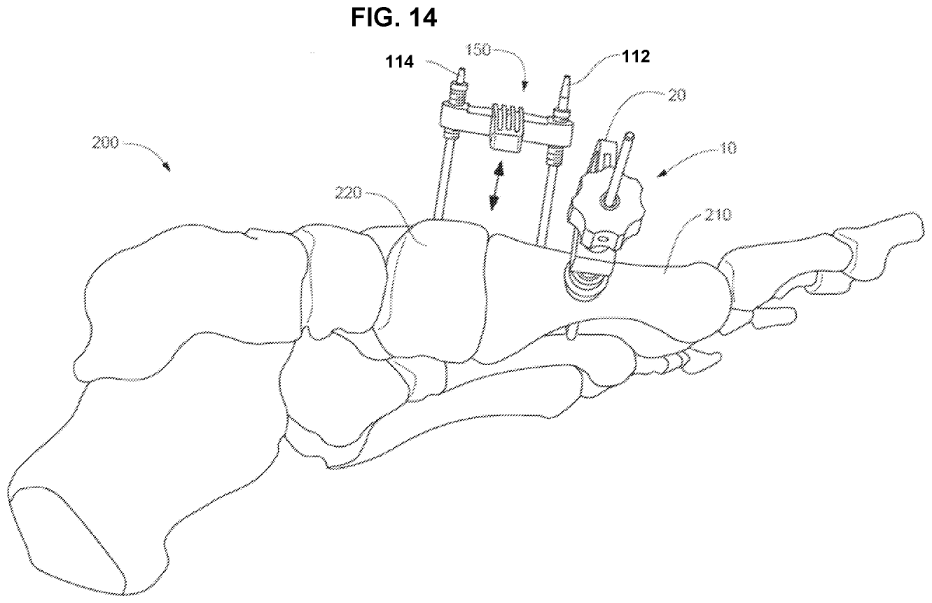

Any angled/converging pins can be removed and the bone preparation guide 150 can be lifted off the substantially parallel first and second pins 112, 114, as shown in FIG. 14. In applications where bone positioning guide 10 is utilized, the bone positioning guide may be removed before or after bone preparation guide 150 is removed. In either case, in some examples, a temporary fixation pin 300 may be used to maintain the position of the underlying bones (e.g., first metatarsal 210 relative to medial cuneiform 222) while bone preparation guide 150 is removed and compressor-distractor 100 is installed.

With the end faces of the metatarsal and cuneiform optionally pressed together via a compressor-distractor, the clinician may provisionally or permanently fixate the bones or bones portions together. For example, one or more bone fixation devices can be applied across the joint and to the two bones to stabilize the joint for fusion, such as two bone plates positioned in different planes. FIG. 14 illustrates an example fixation device arrangement that includes a first bone plate 400 positioned on a dorsal-medial side of the first metatarsal and medial cuneiform and a second bone plate 410 positioned on a medial-plantar side of the first metatarsal and the medial cuneiform. In other embodiments, second bone plate 410 can be a helical bone plate positioned from a medial side of the cuneiform to a plantar side of the first metatarsal across the joint space. The plates can be applied with the insertion of bone screws. Example bone plates that can be used as first bone plate 400 and/or second bone plate 410 are described in US Patent Publication No. US2016/0192970, titled “Bone Plating System and Method” and filed Jan. 7, 2016, which is incorporated herein by reference. Other types in configurations of bone fixation devices can be used, and the disclosure is not limited in this respect. Any temporary fixation pins 300 inserted into the bones can be removed once permanent fixation is applied.

Multi-diameter fixation pins, along with associated techniques and systems, have been described. In some examples, a multi-diameter fixation pin according to the disclosure is included in a disposable, sterile kit that includes associated surgical instrumentation, such as bone positioning guide and/or a preparation guide described herein. Other components that may be included within the sterile kit include bone fixation devices, bone fixation screws, single diameter pins for insertion into pin-receiving holes, and the like.

Various examples have been described. These and other examples are within the scope of the following claims.

1. A method comprising:
inserting a pin having a first diameter into a collet of a powered driver and driving the pin into a bone portion; and
inserting a driver engagement portion of a multi-diameter fixation pin into the collet of the powered driver and driving a bone insertion portion of the multi-diameter fixation pin into the bone portion;
wherein the collet of the powered driver is sized to receive the pin having the first diameter and the driver engagement portion of the multi-diameter fixation pin but not the bone insertion portion of the multi-diameter fixation pin.

2. The method of claim 1, wherein the bone portion is a first bone portion, and inserting the pin having the first diameter into the collet of the powered driver and driving the pin into the bone portion comprises:
inserting a first pin having the first diameter into the collet of the powered driver and driving the pin into the first bone portion,
removing the first pin from the collet of the powered driver while the first pin remains in the first bone portion,
inserting a second pin having the first diameter into the collet of the powered driver and driving the pin into a second bone portion separated from the first bone portion by (a) a joint or (b) a cut dividing a bone into the first bone portion and the second bone portion; and
removing the second pin from the collet of the powered driver while the second pin remains in the second bone portion.

3. The method of claim 1, wherein inserting the driver engagement portion of the multi-diameter fixation pin into the collet of the powered driver comprises inserting the multi-diameter fixation pin into the collet to a depth less than or equal to a depth denoted by an indicator on the multi-diameter fixation pin.

4. The method of claim 3, wherein the indicator comprises a notch formed into a perimeter surface of the body.

5. The method of claim 1, wherein the first diameter and a diameter of the driver engagement portion each ranges from 2.0 mm to 3.2 mm, and a diameter of the bone insertion portion ranges from 0.7 mm to 1.8 mm.

6. The method of claim 1, wherein the first diameter and a diameter of the driver engagement portion each are greater than or equal to 2.0 mm, and a diameter of the bone insertion portion is less than 2.0 mm.

7. The method of claim 1, wherein driving the bone insertion portion of the multi-diameter fixation pin into the bone portion comprises driving the bone insertion portion of the multi-diameter fixation pin through the bone portion and into an adjacent bone portion.

8. The method of claim 1, wherein the bone portion comprises a metatarsal or a portion thereof.

9. The method of claim 8, further comprising moving the bone portion to correct a bunion deformity.

10. The method of claim 9, wherein moving the bone portion to correct the bunion deformity comprises moving the bone portion in a transverse plane and rotating the bone portion in a frontal plane.

11. The method of claim 8, wherein the metatarsal is a first metatarsal.

12. A surgical fixation wire comprising:
a body extending from a first end to a second end opposite the first end, the body defining a driver engagement portion having a first diameter and a bone insertion portion having a second diameter less than the first diameter,
the first end of the body defining a tip configured for insertion into a bone, and
the driver engagement portion being configured for insertion into a powered driver.

13. The fixation wire of claim 12, wherein the body further defines a taper portion between the driver engagement portion and the bone insertion portion, the taper portion having a diameter that transitions from the first diameter to the second diameter.

14. The fixation wire of claim 13, wherein the taper portion tapers at an angle ranging from 10 degrees to 60 degrees.

15. The fixation wire of claim 12, wherein the first diameter ranges from 2.0 mm to 3.2 mm and the second diameter ranges from 0.7 mm to 1.8 mm.

16. The fixation wire of claim 12, wherein the first diameter is greater than or equal to 2.0 mm and the second diameter is less than 2.0 mm.

17. The fixation wire of claim 12, wherein the body further comprises an indicator positioned at a location indicating a depth to which the driver engagement portion can be insertion into the powered driver.

18. The fixation wire of claim 17, wherein the indicator comprises a notch formed into a perimeter surface of the body.

19. The fixation wire of claim 12, wherein the body is a unity body formed of a single piece material.

20. A method comprising:
inserting a driver engagement portion of a multi-diameter fixation pin into a collet of a powered driver and driving a bone insertion portion of the multi-diameter fixation pin through at least one bone portion,
wherein the collet of the powered driver is sized to receive the driver engagement portion of the multi-diameter fixation pin but not the bone insertion portion of the multi-diameter fixation pin.
